(19) United States
(12) Patent Application Publication
(10) Pub. No.: US 2026/0105864 A1
(43) Pub. Date: Apr. 16, 2026

(54) INTRANASAL DRUG DELIVERY DEVICE SIMULATOR

(71) Applicant: Marimo Laboratories LLC, Cambridge, MA (US)

(72) Inventor: Vincent Ted Liao, Cambridge, MA (US)

(21) Appl. No.: 19/357,540

(22) Filed: Oct. 14, 2025

Related U.S. Application Data
(60) Provisional application No. 63/707,512, filed on Oct. 15, 2024.

(51) Int. Cl. G09B 23/28; A61M 15/08

(52) U.S. Cl. CPC G09B 23/28; A61M 15/08

(57) ABSTRACT

Provided is an intranasal medication delivery simulator for training purposes. The simulator comprises a housing, a detachable stopper, and a resettable plunger. The stopper features an internal groove and/or a stopping interface configured to secure the plunger, providing realistic haptic feedback during upward movement when the plunger leaves the groove. The plunger is reset into the groove during downward movement for subsequent use. The simulator may include one or more flexible arms that terminate in a tip, with the groove and/or the stopping interface shaped to correspond to the tip. The housing and stopper may be made from rigid or semi-rigid materials, and the stopper may include radially-projecting fins that are removable or interchangeable. The stopper and plunger are arranged coaxially within the housing, and the stopper may be mounted using a threaded interface, friction-fit, or adhesives. This design allows for realistic, repeatable training without expending medication.

### CROSS-REFERENCE TO RELATED APPLICATIONS

This application is claims priority benefit, including under 35 U.S.C. § 119(e), of U.S. Provisional Patent Application 63/707,512, filed Oct. 15, 2024, which is incorporated herein by reference in its entirety.

### TECHNICAL FIELD

The present disclosure relates to simulators for medical training, specifically an intranasal medication delivery simulator. The disclosure simulates the physical and functional properties of liquid nasal spray drug delivery devices (LNS DDD) for training purposes, providing realistic haptic feedback to users. The simulator is designed to replicate key operational aspects of intranasal drug delivery systems used in emergency medical situations.

### BACKGROUND

Medical emergencies often occur unexpectedly, requiring immediate action. Proper training is critical for improving performance in such high-stress situations. The use of simulators in training enhances both readiness and proficiency in responding to emergencies.

Certain emergency medications are administered using single-use liquid nasal spray (LNS) drug delivery devices (DDD). For instance, naloxone is used for opioid overdose, while midazolam and diazepam are used for treating cluster seizures. Epinephrine is used for anaphylactic allergic reactions. The intranasal route allows for the rapid and non-invasive delivery of lifesaving medications by caregivers and bystanders without the need for needles.

These drug delivery devices are designed to administer a single dose of medication and are intended for use by laypersons or first responders in high-pressure situations. However, errors in administration can lead to a failure in delivering the medication. Furthermore, these devices are sealed in packaging that should not be opened until needed, which limits users' familiarity with the device. Additionally, these devices cannot be tested without expending the single dose, and once used, the DDD must be discarded. Therefore, a reusable simulator is required for training purposes.

LNS DDD typically consist of three main components: a housing for gripping the device, a tapered cylindrical nozzle for insertion into the patient's nostril, and a plunger arranged coaxially with the nozzle. The plunger contains a drug vial sealed with a rubber stopper, positioned adjacent to an internal needle secured by the nozzle. When the user moves the plunger from a first position to a second position by applying pressure, the needle pierces the rubber stopper, dispensing the liquid medication through the nozzle as an intranasal spray.

An ideal simulator would replicate key characteristics of the DDD, such as size, shape, weight, and the haptic feedback experienced during drug dispensing. However, the simulator should also be distinguishable in other aspects to prevent confusion with the actual DDD during an emergency.

Current commercially available simulators closely resemble the LNS DDD in terms of size, shape, and color. One version uses an identical housing and nozzle but replaces the drug vial-containing plunger with a spring-loaded plunger. While this simulates the plunger movement from a first to second position, it does not accurately replicate the haptic feedback of the actual LNS DDD. Another version uses a spring-loaded twisting mechanism to prepare the device for re-use. This adds complexity of manufacturing and use.

### BRIEF DESCRIPTION OF DRAWINGS

FIG. 1 illustrates a front perspective view of the exemplary simulator, showcasing the stopper, plunger, and housing, which replicate certain physical and functional characteristics of a liquid nasal spray drug delivery device (LNS DDD).

FIG. 2 is a rear perspective view of the simulator shown in FIG. 1, providing a detailed look at the back and underside of the housing, as well as the arrangement of the stopper and plunger components.

FIG. 3 is a front view of the simulator depicted in FIG. 1, focusing on the alignment and arrangement of the plunger and stopper within the housing, offering a direct view of the frontal configuration.

FIG. 4 is a side view of the simulator from FIG. 1, showing the profile of the simulator and the relative positioning of the stopper, plunger, and housing.

FIG. 5 is a top view of the simulator shown in FIG. 1, offering an overhead look at the overall shape, and showing how the plunger, stopper, and housing are positioned from above.

FIG. 6 is a bottom view of the simulator shown in FIG. 1, providing a view of the underside, further highlighting the arrangement and design of the simulator's key elements, including the base of the housing and stopper.

FIG. 7 illustrates a front cross-sectional view of the simulator, showing the internal components, including the flexible arms of the plunger, the formed tip, and the stopper's internal profile., which provide feedback during the compression of the plunger.

FIG. 8 is a back-top-left perspective cross-sectional internal view of a stopper, according to another embodiment of the present disclosure, illustrating the stopper's wide nozzle section, the lower portion, the internal cavity with indentations, and the internal stopping interface configured to limit downward travel of the plunger.

FIG. 9 is a rear elevational view of the stopper cross-section of FIG. 8.

FIG. 10 is a front-top-right perspective view of a plunger removed from the stopper, according to another embodiment of the present disclosure, showing a pair of flexible arms terminating in a tip, with outward protrusions configured to engage the stopping interface of the stopper shown in FIG. 8.

FIG. 11 is a front elevational view of the plunger of FIG. 10.

FIG. 12 is a rear elevational view of the plunger of FIG. 10.

FIG. 13 is a back-top-right perspective view of the simulator with the stopper cross-section of FIG. 8 and the Plunger of FIG. 10.

FIG. 14 is a rear elevational view of the simulator with the stopper cross-section of FIG. 8 and the Plunger of FIG. 10.

### SUMMARY

In some embodiments, the intranasal medication delivery simulator comprises a housing and a stopper, the stopper being detachably mounted to the housing. A resettable plunger is insertable into the stopper, and the stopper includes an internal surface with a groove designed to secure the plunger. Movement of the plunger upward causes it to leave the groove, providing realistic haptic feedback during operation, while downward movement resets the plunger into the groove for subsequent use.

The plunger may comprise a tip at its end, and the groove may have a shape corresponding to the tip's shape. The plunger may further include one or more flexible arms terminating at the tip, with these arms potentially being made from plastic, metal, or a combination of materials. The groove may also be formed with one or more indentations or protrusions. In some embodiments, the plunger may feature a tip at the distal end and a grip with a flat surface at the proximal end.

The housing may be constructed from rigid or semi-rigid materials such as plastic, resin, metal, or combinations thereof. The stopper may have one or more radially-projecting fins, which may be removable or interchangeable. The plunger may have a flattened or rounded face, and in some cases, it may also include an indented face. In other embodiments, the stopper and plunger are arranged coaxially within the housing. The stopper may be detachably mounted to the housing using a threaded interface or secured by a friction-fit or adhesives.

In certain embodiments, the stopper is formed with a groove that corresponds to the shape of the plunger tip, securing the plunger and providing haptic feedback during upward movement, while resetting the plunger during downward movement. The plunger may also feature a grip with a flat surface at its proximal end and one or more flexible arms terminating at the tip.

In some embodiments, provided is an intranasal medication delivery simulator, comprising: a housing; a stopper comprising a narrow top section that flares as it extends downward and is detachably mounted to the housing; and a resettable plunger insertable into the stopper, wherein the stopper further comprises an internal surface with an indentation that is configured to secure the plunger, wherein the stopper further comprises one or more radially-projecting fins, wherein movement of the plunger upward causes the plunger to leave the indentation, thereby providing realistic haptic feedback during the movement, and wherein movement of the plunger downward resets the plunger into the indentation for subsequent use.

The plunger may include a tip at an end of the plunger.

The indentation may have a shape that corresponds to a shape of the tip.

The plunger may further include one or more flexible arms that terminate in the tip.

The one or more flexible arms may be formed from a material comprising plastic, metal or a combination thereof.

The indentation may be formed as one or more indentations or protrusions.

The plunger may include a tip at a distal end of the plunger and a grip with a flat surface at a proximal end of the plunger.

The housing may be formed from a rigid or semi-rigid material comprising plastic, resin, metal, or a combination thereof.

The one or more fins may taper as they extend toward the housing.

The one or more fins may be removable or interchangeable.

The plunger may include a flattened or rounded face.

The plunger may further include an indented face.

The stopper and plunger may be arranged coaxially within the housing.

The stopper may be detachably mounted to the housing using a threaded interface.

The stopper may be friction-fit or secured with adhesives to the housing.

In some embodiments, provided is an intranasal medication delivery simulator, comprising: a housing; a stopper mounted to or formed integrally with the housing and defining an internal cavity that comprises: an annular groove having at least one indentation lying in a first diametral plane, and a radially inward stopping interface lying in a second diametral plane that is orthogonal to the first diametral plane; and a plunger slidably disposed within the internal cavity and movable along a longitudinal axis between an extended position and an upward compressed position, the plunger comprising two flexible arms terminating in two corresponding tips, the tips each comprising: a first protrusion extending radially outward in the first diametral plane, and a second protrusion extending radially outward in the second diametral plane; wherein, in the extended position, the first protrusion engages with the indentation to form a primary detent and the second protrusion engages with the stopping interface, thereby preventing withdrawal of the plunger from the stopper; and when the plunger is moved toward the upward distractive position, the first protrusion disengages with the indentation and the second protrusion disengages with the stopping interface.

When the plunger is moved toward the upward compressed position, (i) the flexible arms may elastically deflect to release the first protrusion from the indentation, and (ii) the first protrusion may travel past the groove into a cylindrical pocket of the cavity.

The stopper may comprise a wide top section that tapers as it extends downward and is detachably mounted to the housing.

The simulator of claim 16 may further comprise a lubricant deposited into a upper region of the internal cavity of the stopper.

The lubricant may further comprise wax, silicone grease or PTFE powder.

### DETAILED DESCRIPTION OF EMBODIMENTS

The present disclosure relates generally to medical devices, and more specifically to simulators designed for training purposes in the administration of intranasal medication. In particular, this disclosure pertains to a reusable training device that simulates the physical and functional aspects of liquid nasal spray drug delivery devices (LNS DDD) used for emergency treatments such as opioid overdose reversal and seizure management. This disclosure addresses the need for safe and cost-effective training tools that allow users to practice the administration of medication without the risk of wasting actual medication or causing harm to a patient.

This disclosure is applicable in a wide range of medical training environments, including hospitals, clinics, first responder programs, and public health initiatives. By providing realistic haptic feedback and simulating key features of actual drug delivery devices, this disclosure enhances training outcomes and preparedness for high-stress medical situations. The embodiments described herein represent several preferred configurations, but it should be understood that the disclosure is not limited to the specific structures and methods described, as other variations and equivalents are possible within the scope of the claims.

The features and advantages of this disclosure will be more fully understood from the detailed description of the preferred embodiments and the accompanying drawings, which illustrate the structure and operation of the disclosure. While specific examples are provided, the disclosure is not confined to these embodiments, but rather encompasses all variations, modifications, and improvements that fall within the spirit and scope of the claims appended hereto.

The terminology used herein is for the purpose of describing particular embodiments only and is not intended to be limiting of the disclosure. As used herein, the singular forms “a,” “an” and “the” are intended to include the plural forms as well, unless the context clearly indicates otherwise. These terms are merely intended to distinguish one component from another component, and the terms do not limit the nature, sequence or order of the constituent components. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Throughout the specification, unless explicitly described to the contrary, the word “comprise” and variations such as “comprises” or “comprising” will be understood to imply the inclusion of stated elements but not the exclusion of any other elements.

The term “housing” used herein refers to the structural component of the simulator that encloses or supports other elements such as the stopper and plunger. The housing provides a grip for the user and is designed to mimic the size, shape, and handling of an actual liquid nasal spray drug delivery device (LNS DDD).

The term “stopper” used herein refers to a component that simulates the nozzle of an LNS DDD. The stopper may include features such as radially-projecting fins, grooves, or other distinguishing characteristics to prevent confusion with actual drug delivery devices. The stopper also provides an interface for the plunger and may include internal surfaces to guide and secure the plunger during operation.

The term “plunger” used herein refers to a movable component designed to replicate the actuation of a drug delivery device. The plunger is positioned within the stopper and is configured to move between an extended and a retracted position. The plunger may include flexible arms and tips that interact with internal grooves or recesses in the stopper to provide haptic feedback during use. The plunger is resettable for repeated simulations.

The term “arms” used herein refers to flexible extensions attached to the plunger that terminate in a tip. These arms are designed to engage with the internal features of the stopper, such as grooves or recesses, to provide haptic feedback. The arms may be formed from materials such as plastic or metal, and their flexibility allows for compressive and distractive movement during operation.

The term “groove” used herein refers to an internal recess or channel within the stopper designed to engage with the plunger, providing resistance and realistic haptic feedback during operation. Movement of the plunger within the groove simulates the activation of an actual drug delivery device.

The term “fins” used herein refers to radially-projecting structures that may be included on the stopper to provide visual and tactile distinction between the simulator and a real drug delivery device. The fins may vary in size, shape, and configuration and can be removable or interchangeable to adapt the simulator for different training scenarios.

The term “haptic feedback” used herein refers to the tactile response provided by the simulator to mimic the resistance and sensation experienced during the actuation of a real drug delivery device. This feedback is achieved through the interaction of the plunger with internal components such as grooves, tips, and flexible arms.

The term “resettable” used herein refers to the capability of the plunger to return to its original extended position after being depressed or retracted, allowing for multiple uses of the simulator without the need for external resetting mechanisms.

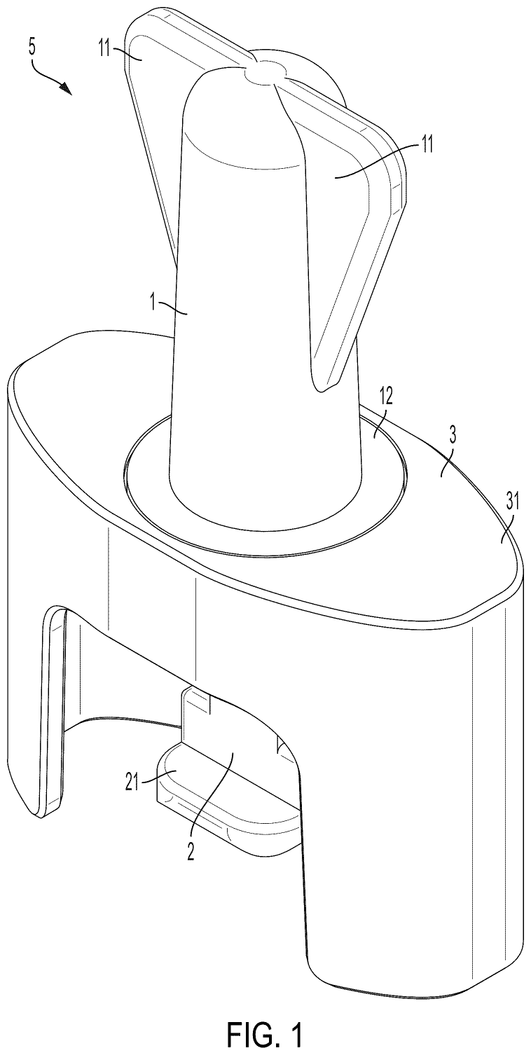

FIG. 1 shows a front perspective view of an exemplary simulator 5. The simulator 5 consists of three primary components: a stopper 1, a plunger 2, and a housing 3. Each of these components is meticulously designed and configured to replicate key physical and functional characteristics of a real liquid nasal spray drug delivery device (LNS DDD). The materials used for constructing these components may vary depending on the specific embodiment, but they are generally made from robust materials such as rigid or semi-rigid plastics, resin, metal, or other durable materials. These materials are chosen for their ability to withstand repeated use without degradation, ensuring long-term reliability and consistent performance. The overall construction aims to provide a realistic training experience, closely mimicking the weight, feel, and operation of actual drug delivery devices, allowing users to practice in conditions as close to real-life scenarios as possible.

The stopper 1 is designed to closely mimic the nozzle of a typical LNS DDD, featuring a tapered cylindrical shape that facilitates easy insertion into a simulation model, such as the nostril of a training mannequin. While the stopper replicates the physical characteristics of an actual nozzle sprayer, it does not include an outlet for dispensing a drug, as the simulator is not intended to deliver any medication. In some embodiments, the stopper 1 may also include additional distinguishing features to ensure it is not mistaken for a real LNS DDD during an actual medical emergency. For instance, the stopper 1 may be equipped with radially-projecting fins 11, which can vary in quantity, shape, attachment method, and overall configuration depending on the embodiment. These fins 11 may taper as they extend toward the housing, providing a streamlined appearance. Additionally, the fins 11 may be curved. These fins serve multiple purposes: they provide a clear tactile and visual distinction between the simulator and a real device, and they discourage the insertion of the simulator into a human nostril for sanitary and infection-control considerations. However, in certain configurations, the fins 11 may be omitted, allowing the stopper to be used in specialized training models, such as simulation mannequins or other controlled training environments. This flexible design ensures that the simulator remains versatile, adaptable for various training scenarios, while maintaining its core function of providing realistic practice without the presence of any actual medication. The stopper 1 may engage with the housing 3 via the threaded cylindrical cylinder 12.

The plunger 2 is positioned substantially coaxially with the stopper 1, ensuring proper alignment during operation. It is designed to move between a first extended position and a second retracted position, simulating the mechanics of an actual drug delivery device. The plunger 2 may feature a flattened or rounded face 22 at one end, allowing the user to apply compressive axial force to move the plunger. As the user presses the plunger upward, the motion closely mimics the activation mechanism of a real nasal spray, delivering a realistic and practical training experience. This movement helps trainees become accustomed to the necessary force and tactile sensation required for actual medication administration. Additionally, the plunger 2 may have a flattened or indented face 21 at the opposite end, enabling the user to apply an downward, distractive axial force to return the plunger to its initial extended position, resetting the device for repeated use. This feature is particularly valuable in training scenarios, allowing for multiple practice sessions without the need for resetting external components. This configuration provides a comprehensive simulation, ensuring that users become familiar with the actuation process of nasal drug delivery devices.

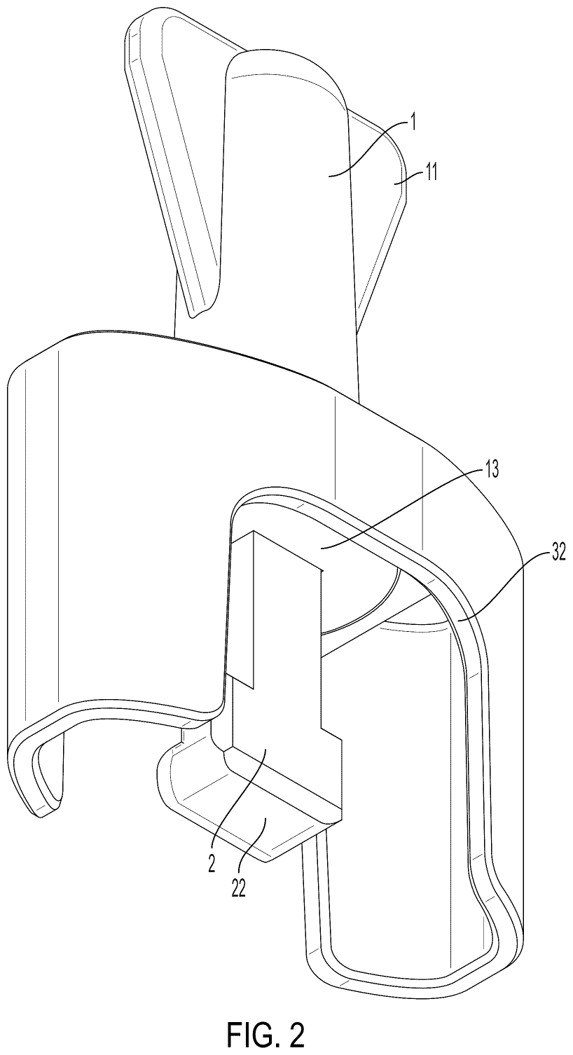

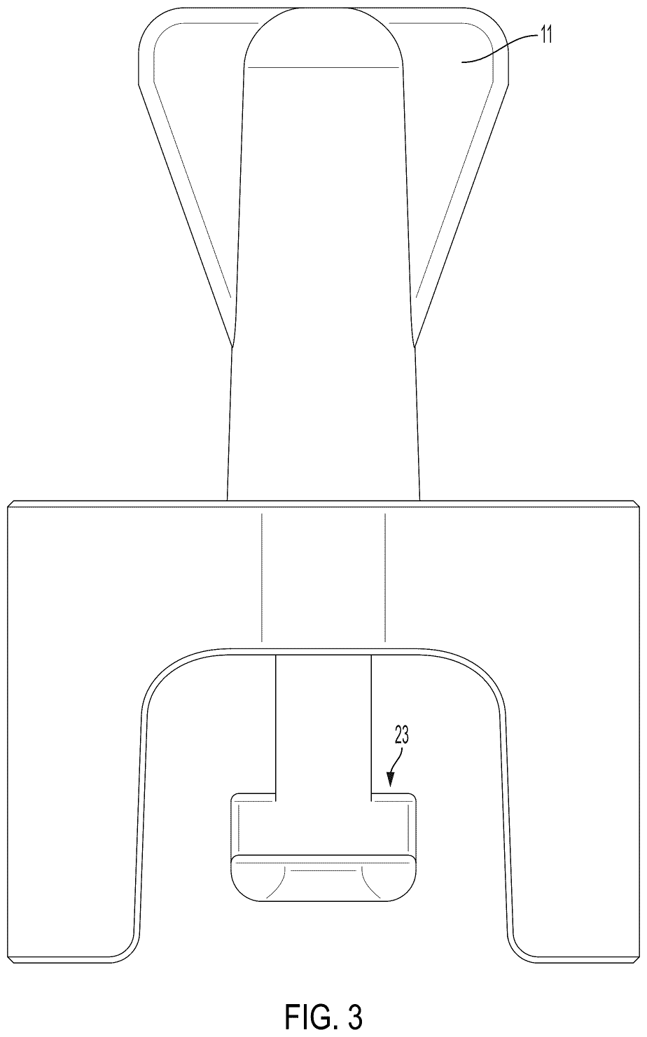

In some embodiments, the plunger 2 may be further equipped with a stopping interface 23, as illustrated in FIG. 3. This interface is designed to interact with an external stopping interface 13 located on the stopper 1, as shown in FIG. 2. The interaction between these two interfaces effectively limits the upward travel of the plunger when compressive force is applied. This built-in stop mechanism prevents the plunger from being pressed beyond a specific point, providing realistic tactile feedback that simulates the natural resistance experienced during actual drug delivery. By incorporating this feature, the simulator closely replicates the physical limitations of a real nasal spray device, ensuring that the user becomes familiar with the proper force and movement required during medication administration. This mechanism not only improves the accuracy of training but also reinforces correct usage by preventing over-compression or improper actuation, which could otherwise lead to user error in real-life scenarios.

The housing 3 is designed to closely resemble the body of a real LNS DDD, ensuring that the user can grip and operate the simulator comfortably and naturally. By mirroring the dimensions and feel of an actual device, the housing provides a realistic training experience. In some embodiments, the housing may include a flat face 31 to enhance the user's grip, ensuring secure handling during operation, especially in high-stress or emergency training scenarios. This design consideration helps users build muscle memory and familiarity with the actual LNS DDD.

The stopper 1 may be detachably mounted to the housing using a variety of attachment mechanisms, depending on the specific embodiment. For instance, the stopper could be secured using a threaded interface 33, which allows for easy removal and reattachment. In other embodiments, the stopper may rely on a friction-fit or adhesive bonding to securely connect it to the housing. In some cases, the stopper 1 may even be monolithically integrated into the housing, forming a single, seamless unit that simplifies the overall design and manufacturing process.

Additionally, the housing 3 may feature a contour 32 that is specifically designed to allow unobstructed operation of the plunger. This ensures that the user can easily compress the plunger without any interference from the housing or surrounding components, making the simulator both user-friendly and highly functional for repeated use in training environments.

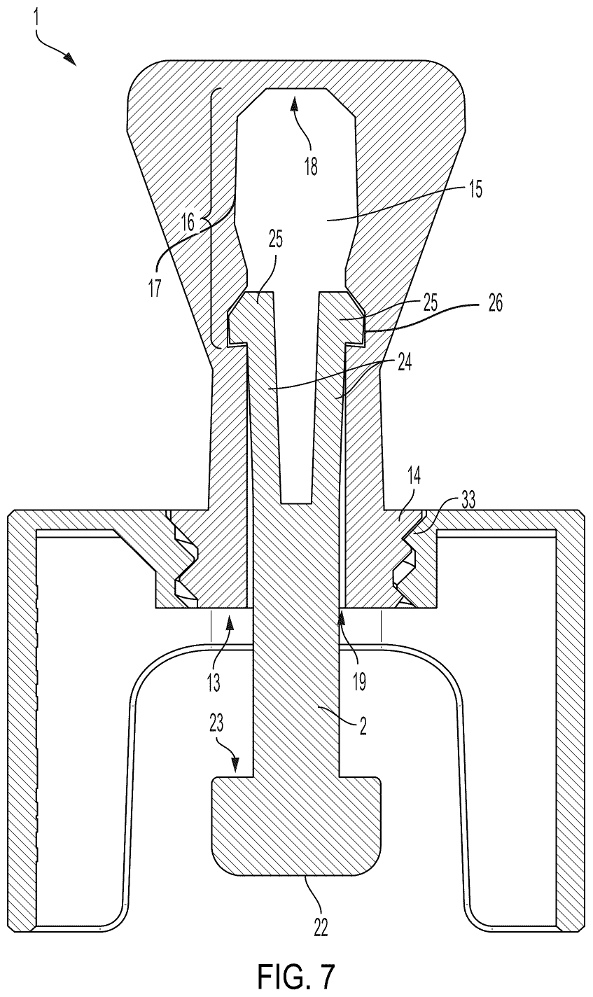

FIG. 7 provides a front cross-sectional view of the simulator 5, illustrating additional internal components that enhance its functionality. In some embodiments, the plunger 2 may be designed with one or more flexible arms 24, each terminating in a formed tip 25. These flexible arms 24 allow the plunger to perform both compressive and distractive movements, while ensuring the tip 25 remains securely positioned within the stopper 1. The internal profile of the stopper 16 may be customized with features such as indentations, protrusions, or a stopping interface 18. These features interact with protrusions 26′ on the tip 25 and arms 24 to deliver realistic haptic feedback as the plunger is compressed. This feedback mimics the tactile sensation experienced during actual drug administration, offering users a realistic and accurate simulation of the device's operation.

In some embodiments, the tips 25 and arms 24 of the plunger are positioned within a groove or recess 17 inside the stopper 1. The geometry of this groove is configured to ensure that the plunger remains securely retained within the stopper during use. For example, the width of the tips 25 and arms 24 may exceed the width of the stopper's entry hole 19, preventing unintended disengagement. The flexible materials used for the arms 24, such as plastics or metals, allow them to temporarily deform when inserted into the stopper entry hole 19. Once inserted, the arms 24 return to their original position, securely locking the plunger within the stopper 1. This feature ensures that the simulator provides a realistic and consistent experience, even after repeated use.

In other embodiments, the stopper 1 may be equipped with more than one groove or recess 17 to create a more refined haptic experience. These multiple grooves may be spaced at different intervals within the stopper, allowing the user to feel a gradual movement as the plunger progresses through each stage of compression. This arrangement can simulate the incremental stages of drug administration, providing distinct tactile feedback at each groove. The graduated movement enhances realism by mimicking the step-by-step sensation experienced during actual drug delivery, further improving the training experience.

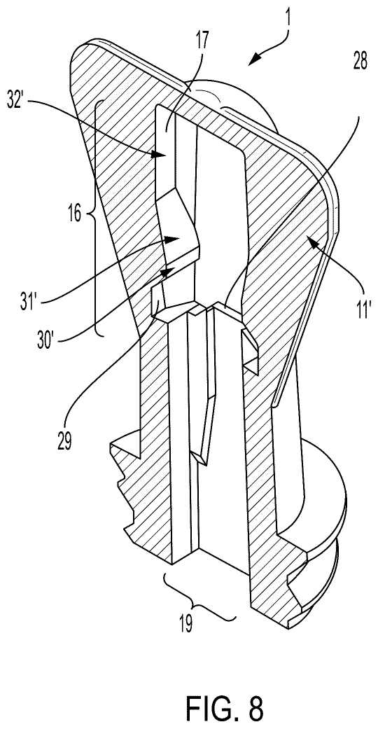

FIG. 8 illustrates the stopper 1′ in isolation and cross-section from a back-top-left perspective. The drawing shows the internal profile of the stopper 16′, the entry hole 19′, and the radially projecting fins 11′. This view of the internal cavity 17′ highlights an annular groove that contains a pair of indentations 29′ lying in a first diametral plane; these indentations are sized and positioned to receive the first protrusions 26′ on the plunger tip 2′, thereby forming a primary dent. Immediately above the indentations 29′ is the bore surface 30′ that guides the first protrusions 26′ upward without any inward taper. The bore surface 30′ then flares outward along a short conical slope 31′ and then transitions into a cylindrical pocket 32′ of substantially uniform diameter.

A radially inward stopping interface 28′, oriented in a second diametral plane orthogonal to the first diametral plane, cooperates with the second protrusions 27′ carried on the plunger's flexible arms.

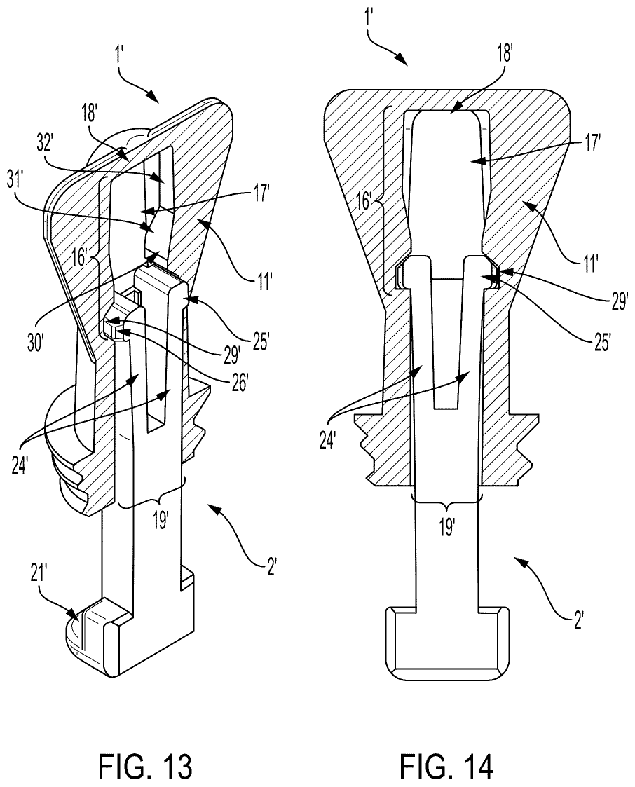

When a user returns the plunger 2′ downward to reset the simulator (as shown in FIG. 13), the first protrusions 26′ leave the cylindrical pocket 32′, descend the flared slope 31′, and snap into the indentations 29′. At the same time, the protrusions 27′ seat against the stopping interface 28′, preventing any further downward travel beyond the indentations 29′ and the interface 28′. This action reestablishes the primary detent and prepares the simulator for the next compression stroke.

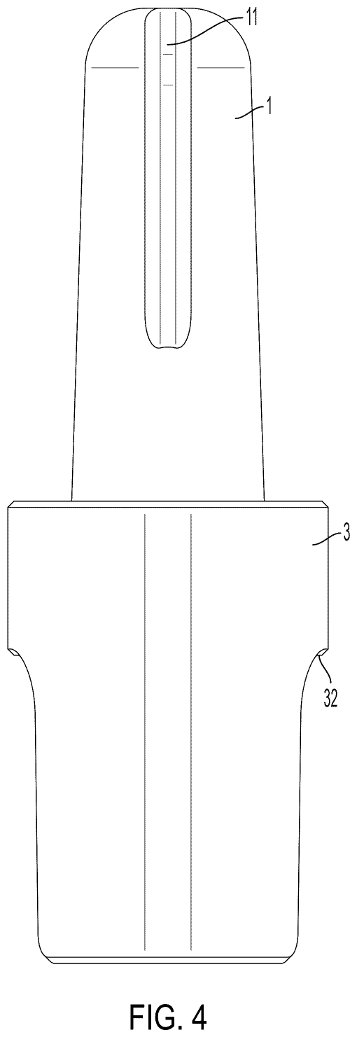

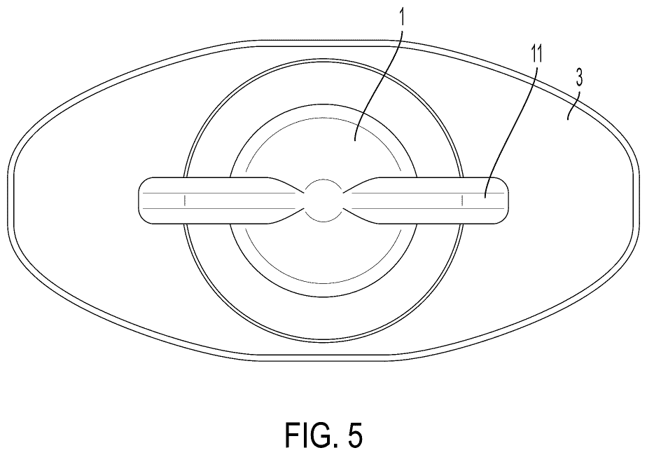

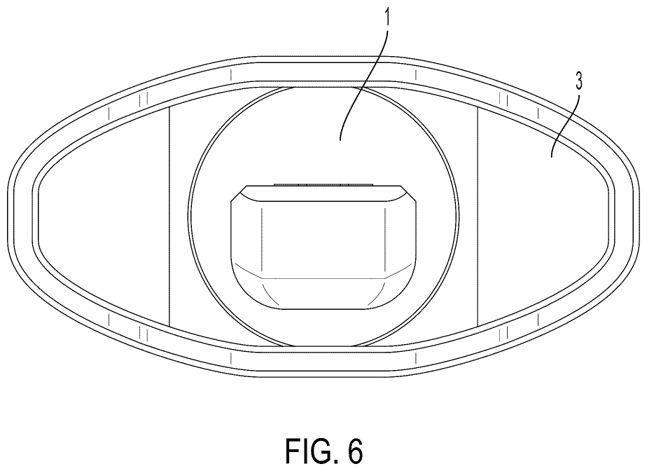

Conversely, when a user pushes the plunger 2′ upward, the first protrusions 26′ disengage from the indentations 29′ climb the bore surface 30′ and the flared slope 31′ to re-enter the cylindrical pocket 32′. Additional upward travel is arrested when the plunger 2′ contacts the internal ceiling surface of the stopper 18′. This two-plane, two-stage retention scheme—indentations 29′ for the first protrusions 25′ and stopping interface 28′ for the second protrusions 27′—differs from the arrangement shown in FIGS. 1-7 and ensures reliable, repeatable training with realistic tactile feedback.

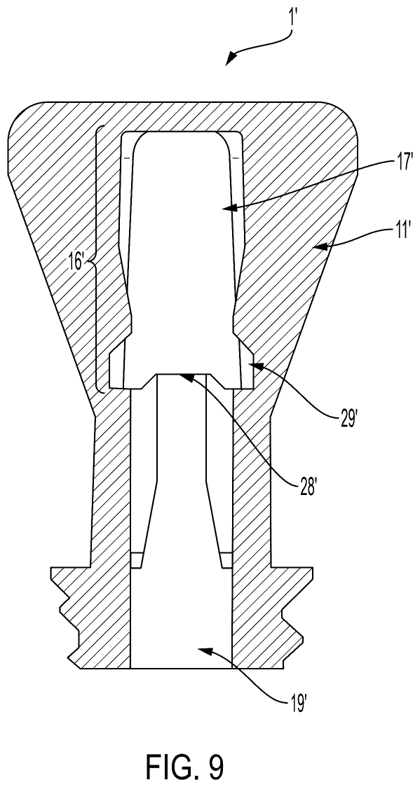

FIG. 9 provides a rear elevational view of the stopper 1′. This orthogonal view clarifies the external outline of the internal profile of the stopper 16′ and the entry hole for the plunger 19′, which is narrower in width than the area of the indentations 29′. The axial alignment of the internal cavity 17′ and location of the stopping interface 28′ and cross-section of the fins 11′ are re-demonstrated.

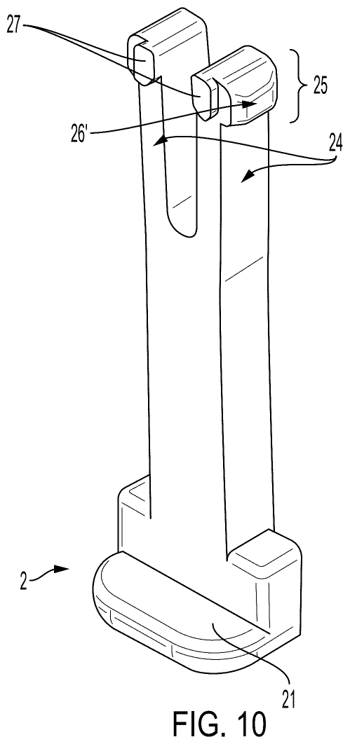

As best observed in FIG. 10, a plunger 2′ terminates in an enlarged tip 25′. A pair of first protrusions 26′ are formed around the periphery of the tip 25′ and extend radially outward in a first diametral plane. Each flexible arm 24 further carries a second protrusion 27′ that also projects radially outward but lies in a second diametral plane orthogonal to the first plane; accordingly, the two sets of protrusions occupy mutually orthogonal planes around the plunger axis. When the plunger 2 is fully seated in the stopper 1 (FIG. 13), the protrusions 26′ lodge within the indentations 29′ to establish the initial detent. To move the plunger from a first ready-to-use position to a second actuated position, an upward compressive force must (i) compress the arms 24′ enough to release the first protrusions 26′ from the indentation 29′ and is then (ii) arrested when the tip 25′ collides with the internal ceiling surface of the stopper 18′ and/or the plunger interface 23′ collides with the stopper at external face 13′. To move the plunger from the second actuated position back to the first ready-to-use position, a downward distractive force compresses arms 24′ to release from the cylindrical pocket 32′, past the flared slope 31′ and back into indentations 29′. The protrusions 26′ interfacing with the indentations 29′ along with the protrusions 27′ seats on the stopping interface 28′ prevent the plunger 2′ from accidentally being pulled out of the stopper 1′. This orthogonal, two-stage arrangement yields enhanced retention without diminishing the crisp break feel experienced during actuation from the first position to the second position.

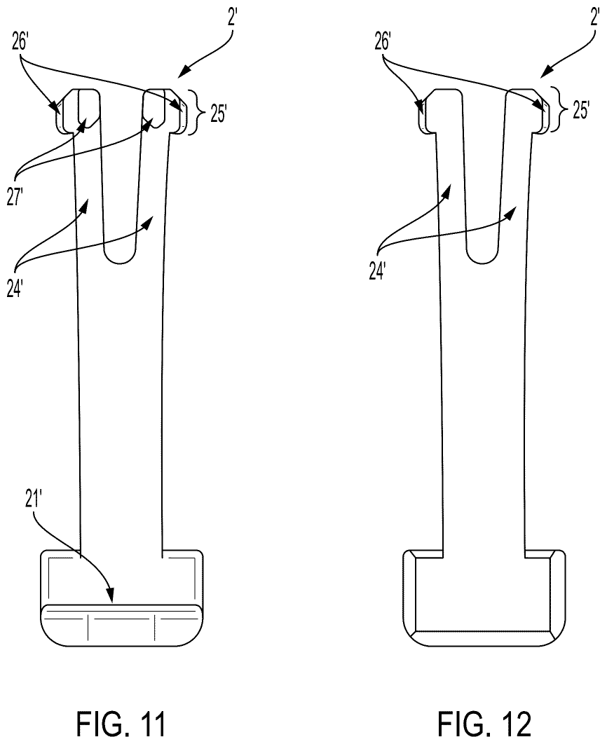

FIG. 11 shows a front elevation view of the plunger 2′, whereas FIG. 12 shows a rear elevational view of the same component. These orthogonal drawings confirm the symmetry of the flexible arms 24′ and illustrate the thickness of the tip 25′ relative to the plunger body. The distal surface 21′ is visible in FIG. 11 and can be engaged by a user's finger to help reset the plunger downward to its extended position after compression.

Turning to FIG. 13, a back-top-right perspective view of the plunger 2′ inserted into a cross-section of stopper 1′, the flexible arms 24′ are shown slightly deflected so that the tip 25′ nests in the groove within cavity 17′. If an upward compressive force is applied, the tip 25′ collides with the ceiling of the cavity 18′ to arrest further movement. During a normal compressive stroke, the arms 24′ deflect inward, allowing the tip 25′ to snap past the indentations 29′ and produce the desired tactile break sensation.

FIG. 14 provides a rear elevational view of plunger 2′ inserted into a cross-section of stopper 1′. This drawing highlights the coaxial alignment of the plunger 2′ within the stopper 1′ and shows the relative positions of the entry hole 19′ and the distal end of the plunger.

In some embodiments, the stopper 1′ may be interchangeable, allowing the simulation of different drug delivery devices. For instance, a stopper could be specifically designed to replicate various types of LNS DDDs, such as those used for opioid reversal, seizure management, or other emergency medications. This modular design provides enhanced versatility, allowing trainers to easily switch between different stoppers for practicing with multiple drug types, all using the same simulator body.

Another embodiment of the simulator may incorporate an audible feedback mechanism. In this design, pressing the plunger generates a sound that mimics the noise typically made during the actual dispensing of medication. This feature adds an additional sensory input for the user, enhancing the realism of the simulation by simulating both the tactile and auditory elements of drug delivery.

In further embodiments, the simulator could be equipped with sensors and designed to interface with a mobile application or a training system. Sensors embedded within the plunger and stopper could measure various parameters, such as the force applied or the accuracy of the simulated drug administration. The data collected could then be transmitted to a mobile device or training software, providing real-time feedback to the user or instructor, thus allowing for more precise assessments of the user's performance during the training session.

In another variant, the simulator may be integrated with virtual or augmented reality (VR/AR) training systems. In such an embodiment, the physical operation of the simulator would be synchronized with a virtual environment, where the user administers the drug to a virtual patient. This immersive experience could be an invaluable tool for training medical professionals and first responders, offering realistic practice scenarios that closely mirror the high-pressure environments they may face in real-life emergencies.

Finally, in some embodiments, the plunger could be designed with adjustable resistance settings, allowing the user to modify the force required to simulate the operation of different drug delivery devices. The resistance can be adjusted in various ways, including altering the materials used for the plunger or stopper. For instance, using a stiffer or more flexible material for the plunger arms or tips can directly influence the amount of force needed to operate the device. Additionally, varying the weight of the simulator components, such as the housing or the plunger itself, can change the resistance experienced by the user.

Another approach is to modify the tension in the plunger's internal components, such as incorporating a spring or elastic mechanism with adjustable tension settings. This could simulate varying levels of compression force required by different drug delivery devices. Altering the shape or flexibility of the plunger tip that interacts with the internal stopper can also affect the frictional resistance, modifying the haptic feedback. These variations enable a more tailored and realistic training experience, accurately reflecting the diversity of drug delivery devices used in emergency medical settings, thus improving both the effectiveness and adaptability of the simulator.

In certain embodiments, one or more small beads or flakes of food-grade paraffin wax—preferably a fully refined paraffin having a melting point of about 70° C. to 75° C.—may be deposited into the upper region of the stopper cavity before the plunger is installed. Actuation of the plunger causes the wax to distribute over the mutually sliding surfaces of the stopper and plunger. The resulting wax coating may measurably lower static and dynamic friction, eliminates squeaking, and preserve the crisp “snap-through” haptic event at the groove for several hundred training cycles without requiring additional maintenance.

The wax may be inert, non-toxic, and easily distinguished from any medicament, ensuring safe handling in classroom or field-training environments. Alternative lubricants may include, but not be limited to, medical-grade silicone grease or PTFE micro-powders.

It should be understood that the foregoing detailed description and accompanying drawings are illustrative and not intended to limit the scope of the disclosure. Various modifications, changes, and substitutions may be made without departing from the spirit and scope of the disclosure as defined by the claims. The disclosure is intended to cover all embodiments that fall within the scope of the claims and their legal equivalents, including those that perform substantially the same function in substantially the same way to achieve the same result.

Additionally, although specific materials, configurations, and operational details have been described herein, those skilled in the art will appreciate that these details can be varied to suit particular use cases without altering the disclosure's fundamental characteristics. All such alternatives, modifications, and equivalents are intended to be included within the scope of the disclosure as defined by the following claims.

Moreover, any features disclosed in the present application may be combined in any manner or configuration that enhances the performance or utility of the disclosure, provided such combinations fall within the bounds of patentable subject matter. The claims are not limited to the specific configurations shown and described, but are intended to encompass all applicable variations that will be readily apparent to those skilled in the art.

What is claimed is:

1. An intranasal medication delivery simulator, comprising:
a housing;
a stopper comprising a narrow top section that flares as it extends downward and is detachably mounted to the housing; and
a resettable plunger insertable into the stopper,
wherein the stopper further comprises an internal surface with an indentation that is configured to secure the plunger,
wherein the stopper further comprises one or more radially-projecting fins,
wherein movement of the plunger upward causes the plunger to leave the indentation, thereby providing realistic haptic feedback during the movement, and
wherein movement of the plunger downward resets the plunger into the indentation for subsequent use.

2. The simulator of claim 1, wherein the plunger comprises a tip at an end of the plunger.

3. The simulator of claim 2, wherein the indentation has a shape that corresponds to a shape of the tip.

4. The simulator of claim 2, wherein the plunger further comprises one or more flexible arms that terminate in the tip.

5. The simulator of claim 4, wherein the one or more flexible arms are formed from a material comprising plastic, metal or a combination thereof.

6. The simulator of claim 1, wherein the indentation is formed as one or more indentations or protrusions.

7. The simulator of claim 1, wherein the plunger comprises a tip at a distal end of the plunger and a grip with a flat surface at a proximal end of the plunger.

8. The simulator of claim 1, wherein the housing is formed from a rigid or semi-rigid material comprising plastic, resin, metal, or a combination thereof.

9. The simulator of claim 1, wherein the one or more fins taper as they extend toward the housing.

10. The simulator of claim 1, wherein the one or more fins are removable or interchangeable.

11. The simulator of claim 1, wherein the plunger comprises a flattened or rounded face.

12. The simulator of claim 11, wherein the plunger further comprises an indented face.

13. The simulator of claim 1, wherein the stopper and plunger are arranged coaxially within the housing

14. The simulator of claim 1, wherein the stopper is detachably mounted to the housing using a threaded interface.

15. The simulator of claim 1, wherein the stopper is friction-fit or secured with adhesives to the housing.

16. An intranasal medication delivery simulator, comprising:
a housing;
a stopper mounted to or formed integrally with the housing and defining an internal cavity that comprises:
(i) an annular groove having at least one indentation lying in a first diametral plane, and
(ii) a radially inward stopping interface lying in a second diametral plane that is orthogonal to the first diametral plane; and
a plunger slidably disposed within the internal cavity and movable along a longitudinal axis between an extended position and an upward compressed position, the plunger comprising two flexible arms terminating in two corresponding tips, the tips each comprising:
(i) a first protrusion extending radially outward in the first diametral plane, and
(ii) a second protrusion extending radially outward in the second diametral plane;
wherein, in the extended position, the first protrusion engages with the indentation to form a primary detent and the second protrusion engages with the stopping interface, thereby preventing withdrawal of the plunger from the stopper; and
when the plunger is moved toward the upward distractive position, the first protrusion disengages with the indentation and the second protrusion disengages with the stopping interface.

17. The simulator of claim 16, wherein when the plunger is moved toward the upward compressed position,
(i) the flexible arms elastically deflect to release the first protrusion from the indentation, and
(ii) the first protrusion travels past the groove into a cylindrical pocket of the cavity.

18. The simulator of claim 16, wherein the stopper comprises a wide top section that tapers as it extends downward and is detachably mounted to the housing.

19. The simulator of claim 16, further comprising a lubricant deposited into a upper region of the internal cavity of the stopper.

20. The simulator of claim 16, wherein the lubricant comprises wax, silicone grease or PTFE powder.
